(19) United States
(12) Patent Application Publication
(10) Pub. No.: US 2021/0307988 A1
(43) Pub. Date: Oct. 7, 2021

(54) PATHOGEN EXAMINATION UNIT

(71) Applicant: Oldcastle BuildingEnvelope, Inc., Dallas, TX (US)

(72) Inventors: Philip Benes, Dallas, TX (US); Conor McDonald, Dallas, TX (US)

(21) Appl. No.: 17/223,975

(22) Filed: Apr. 6, 2021

Related U.S. Application Data
(60) Provisional application No. 63/005,514, filed on Apr. 6, 2020.

(51) Int. Cl. A61G 10/00; A61L 2/07; A61L 2/10; A61M 16/00; A61G 10/02

(52) U.S. Cl. CPC A61G 10/005; A61L 2/07; A61L 2/10; A61M 16/0003; A61M 2205/053; A61L 2202/122; A61L 2202/121; A61L 2202/25; A61G 10/02

(57) ABSTRACT

A pathogen examination unit includes a working side, two non-working sides, and an entrance side. The working side of the pathogen examination unit may include a panel that is positioned between two users, such as between a healthcare worker and a patient. In some cases, the pathogen examination unit places one user, such as the healthcare worker, inside the pathogen examination unit, which reduces the number of surfaces and objects that must be disinfected between patients.

### REFERENCE TO RELATED APPLICATION

This application claims the benefit of U.S. Provisional Patent Application No. 63/005,514, filed on Apr. 6, 2020 and entitled PATHOGEN EXAMINATION UNIT, the content of which is hereby incorporated by reference in its entirety.

### BACKGROUND

In every epidemic and pandemic outbreak, including but not limited to the current COVID-19 coronavirus, other coronaviruses (such as the Middle East Respiratory Syndrome (“MERS”) and Severe Acute Respiratory Syndrome (“SARS”)), Ebola (such as the Ebola, Sudan, Tai Forest, and Bundibugyo species), and influenza (such as influenza A (e.g., H1N1, H3N2, and other strains), influenza B (e.g. Victoria, Yamagata, and other strains), and influenza C) outbreaks, healthcare workers are widely and disproportionately affected as compared to the people they are trying to help. Throughout this document, the term “healthcare worker” is intended to refer to anyone involved in the performance of healthcare services who may be at risk of exposure to pathogens as a result of performing such services including but not limited to doctors, nurses, physician's assistants, nurse practitioners, pharmacists, at-home caregivers, aides, helpers, laboratory technicians, and medical waste handlers. Evidence from past epidemics suggests that healthcare workers are at much higher risk than the general population of being infected, even up to 21-32 times more likely to be infected.

Particularly in response to the recent COVID-19 outbreak, hospitals and clinics around the world are facing challenging decisions and are rapidly adjusting their standard operating procedures to manage the evolving pandemic. Non-clinical space is being re-purposed for patients, and staff are often called upon to serve roles for which they were not hired as a means of filling workforce shortages. The combined effect of increased work hours, the additional stress of increased patient loads, and concern over seeing contagious patients (and potentially placing the healthcare worker's family and friends at risk) can increase burnout.

Once the COVID-19 pandemic slows, the United States (and countries around the world) will need to focus on long-term investments in public health infrastructure. In particular, such investments will need to address the protection of healthcare workers because we cannot protect the public without protecting them. As an added concern, the United States healthcare workforce is aging. Currently, approximately 31% of physicians and 14% of nurses in active practice are 60+ years old, which places them in the “at risk” group for the current COVID-19 coronavirus.

The issues that the healthcare industry are facing with the COVID-19 coronavirus pandemic (and which are not necessarily unique to the COVID-19 coronavirus pandemic) include:
Providing better safeguards for the healthcare workers that are treating and testing patients because the current methods are placing the healthcare workers and their families at risk.
Providing better safeguards for the patients that are being treated and tested because infected patients can infect others, and non-infected patients are vulnerable.
Providing more facilities for the volume of sick people because emergency rooms are overcrowded, intensive care units may be overwhelmed, testing areas are difficult to properly sanitize, and social distancing is difficult.
Providing an alternative to the protective equipment shortages because safety materials are in short or non-existent supply due to, among other things, public consumption, patient volume and wasteful testing processes, and physical space is not sufficient to protect healthcare workers from the many pathogen exposure routes they may encounter (e.g., bloodborne, contact, droplet, or airborne).
Providing improved temporary testing areas that can better protect healthcare workers who are currently exposed, eliminate the wasteful and time-consuming work of relocating testing areas as the pandemic moves, and provide a safe alternative for testing in remote areas where safety equipment may be in short supply or non-existent.
Providing mass testing for more accurate, precise, and reliable data, which is critical to combating spread of the disease.

Efforts have been made in recent weeks to address some of these issues. For example, Yangji Hospital in South Korea created a testing unit based on biosafety cabinets (also known as isolation glove boxes), where healthcare workers are shielded from patients by a transparent plastic booth through which arm-length rubber sleeves with gloves are inserted to allow for physical interaction with the patient. This design places the patient inside the booth and the healthcare worker on the outside. This arrangement allows healthcare workers to examine patients from behind the safety of a plastic panel. Each patient steps into the booth for a rapid consultation by intercom with a healthcare worker who, if necessary, obtains a specimen from the patient by swabbing the patient's nose and throat using the arm-length rubber gloves built into the panel. The design minimizes direct contact between potentially infected patients and the healthcare worker and also shortens the time of testing. These booths use negative air pressure to prevent pathogens (including, but not limited to, airborne pathogens generated by patient sneezing) from escaping outside of the booth. While confining pathogens to the interior of the booth is beneficial, it also means that the booth must be sufficiently disinfected after each patient in order to avoid exposure to subsequent patients.

A shortcoming of the aforementioned design is that, due to the time it takes to disinfect the booth between patients, the throughput rate of patients tested is much lower than what is needed in a mass testing situation. With this design, the whole process takes about 25 minutes (6-7 minutes per person, plus 18-19 minutes to clean the booth) because all surfaces inside the booth must be thoroughly disinfected and the air sufficiently ventilated or otherwise cleaned before the next patient can step inside. As a result, the design has a throughput rate of only 2-3 patients/hour. In terms of throughput rate, the design is not a significant improvement over traditional virus testing, which takes 30 minutes per patient. Furthermore, while the healthcare workers are protected from direct infection by the patient inside the booth, the healthcare workers are still required to wear safety equipment to protect themselves from the environment outside the booth.

To improve the speed of the process, a South Korean company developed a rapid walk-through booth. This booth is made by inverting the existing walk-through booth testing system described above to place the healthcare worker inside the booth. In doing so, the total test time was reduced to approximately 15 minutes per patient, thereby increasing throughput to 4 patients per hour. The revised booths are designed for outdoor use where there is less risk of infection between patients because the patients will be able to maintain a sufficiently safe distance from one another while they are waiting in line.

By placing the healthcare worker inside the booth, it is more critical that the booths are well-sealed to prevent pathogens from entering the booth. Furthermore, in both of the aforementioned designs, the panels are not formed of a robust material that can withstand continued cleaning without becoming scratched and hazy. These booths are also not shipped in a panelized (i.e., disassembled stackable) form that can be easily assembled on site by unskilled labor, a key feature that will further aid in the rapid distribution, transportation, and mobilization across geographies in response to pandemic spread.

### BRIEF DESCRIPTION

The embodiments described herein address the shortcomings present in both of the previous designs. Certain embodiments place the healthcare worker inside the pathogen examination unit, which reduces the number of surfaces and objects that must be disinfected between patients. Such embodiments may be referred to as having a “healthcare worker interior,” while embodiments in which the patient is placed within the booth may be referred to as having a “patient interior.”

All embodiments incorporate the tight joinery used on window wall and curtain wall products to minimize air flow. The tight seal minimizes the potential exchange of pathogen-contaminated air, which allows the healthcare worker to remain in a clean and safe environment without the need for cumbersome (and dwindling supply of) personal protective equipment (respirator, hazmat suit, etc.)—a precaution that is still necessary in the previous designs. In the embodiments having a healthcare worker interior, the venting mechanism for the unit can be setup to positively pressurize the interior to eliminate any unwanted air exchange from exterior to interior, thus ensuring zero air flow from the outside (unclean) to the inside (clean) space.

The unit is also reversible, such that the unit may be arranged to place the patient inside the unit with the integral venting mechanism for the unit setup to negatively pressurize the interior to eliminate any unwanted air exchange from interior to exterior, thus ensuring zero air flow from the inside (unclean) to the outside (clean) space. In addition, the units are modular in that they can be arranged adjacent to one another, allowing both a healthcare worker interior and a patient interior. In other words, the healthcare worker and the patient are able to interact with each other from within separate booths, i.e., a positively pressurized booth for the healthcare worker and a negatively pressurized booth for the patient. This feature makes the units highly adaptive to the varying and particular pathogenic concerns that accompany different infectious diseases.

The healthcare worker may interact with the patient (e.g. collect samples, take vitals, speak, etc.) via electronic communication systems, sealed gloves, medical devices and other products (e.g., stethoscope, sphygmomanometer, tongue depressor, swab, syringe, etc.), or any other means. The patient may also interact with the healthcare worker via electronic communication, they may interact with one another through various other means (e.g., facial and emotional expressions, body language, physical reactions, etc.) that may be more informative to the healthcare worker and comforting to the patient.

By reducing the area of surfaces to be cleaned (in the configurations having a healthcare worker interior) and work required to clean the surfaces (regardless of the configuration), the throughput of patients may be doubled or tripled. Furthermore, the unit is a turnkey solution, incorporating, storing and protecting all materials and equipment needed to assemble the unit. For example, the unit may be provided with glovebox sleeves that are sealed and secured to glovebox ports, shoulder length disposable gloves (such as Nitrile or other non-latex gloves where possible) that may be positioned over the glovebox sleeves, and two-way communication devices, and may further be provided with a stethoscope, non-contact thermometer, hand sanitizer, and a hand sanitizer dispenser.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIGS. 1-3 perspective views of a pathogen examination unit, according to certain embodiments of the present invention.

FIG. 4 is a perspective views of multiple pathogen examination units of FIGS. 1-3 that are not connected to one another.

FIG. 5 is a perspective view showing an arrangement of the pathogen examination unit of FIGS. 1-3 with a patient staging area.

FIG. 6 is a perspective view of a sealed two-way communication device that may be combined with the pathogen examination unit of FIGS. 1-3.

FIG. 7 is a front view of the pathogen examination unit of FIGS. 1-3 with a collection drawer and container attached to the working side.

FIG. 8 is a front view of the pathogen examination unit of FIGS. 1-3 with a collection bin and container attached to the working side.

FIG. 9 is a front view of the pathogen examination unit of FIGS. 1-3 with a receptacle and container attached to the working side.

FIG. 10 is a front view of the pathogen examination unit of FIGS. 1-3 with a funnel and container attached to the working side.

FIG. 11 is a perspective view of a collection drawer that may be combined with the pathogen examination unit of FIGS. 1-3.

FIG. 12 is a perspective view of a set of collection bins.

FIG. 13 are front views of sides of the pathogen examination unit of FIGS. 1-3.

FIG. 14 is a cross-sectional view as seen from above of the pathogen examination unit of FIGS. 1-3.

FIG. 15 is a cross-sectional view as seen from the front of the pathogen examination unit of FIGS. 1-3.

FIG. 16 is a partial view of a panel joined to a structural component of the pathogen examination unit of FIGS. 1-3.

FIG. 17 is a view of the upper surface of the pathogen examination unit of FIGS. 1-3.

FIG. 18 is a view of the bottom surface of the pathogen examination unit of FIGS. 1-3.

FIGS. 19-31 are various views of structural components that may be used to construct the pathogen examination unit of FIGS. 1-3.

FIG. 32 illustrates an assembly method of the pathogen examination unit of FIGS. 1-3 from a panelized shipment.

### DETAILED DESCRIPTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, and, as such, can, of course, vary. It is also to be understood that the terminology used herein is for describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to “a band” can include two or more such bands unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance can or cannot occur, and that the description includes instances where said event or circumstance occurs and instances where it does not.

The word “or” as used herein means any one member of a particular list and includes any combination of members of that list. Further, one should note that conditional language, such as, among others, “can,” “could,” “might,” or “can,” unless specifically stated otherwise, or otherwise understood within the context as used, is generally intended to convey that certain aspects include, while other aspects do not include, certain features, elements, and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular embodiment. Directional references such as “up,” “down,” “top,” “left,” “right,” “front,” “back,” and “corners,” among others are intended to refer to the orientation as illustrated and described in the figure (or figures) to which the components and directions are referencing.

Unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

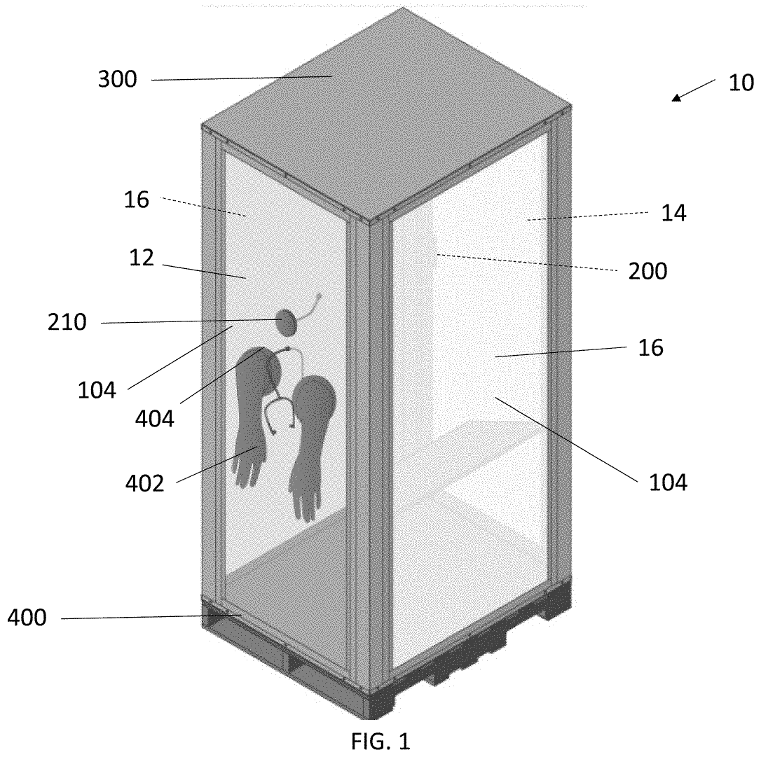

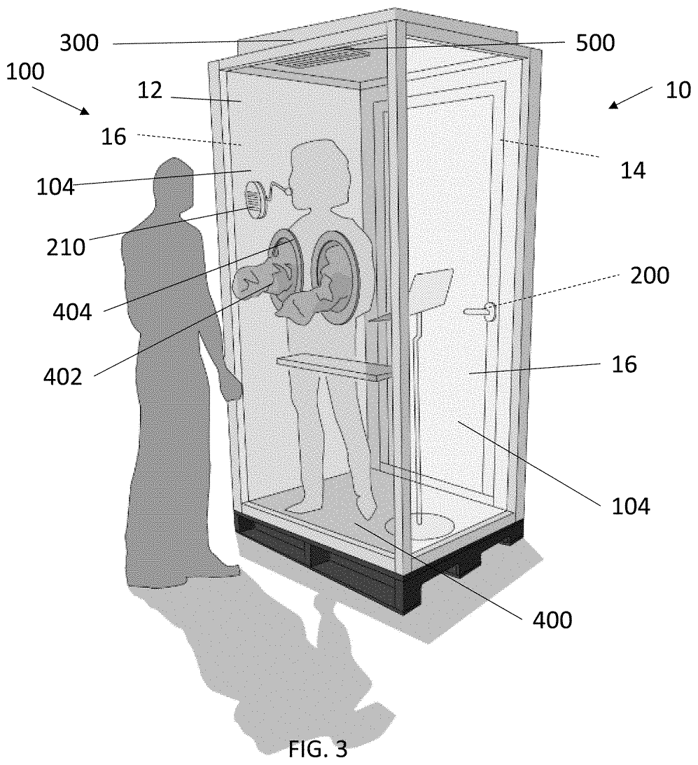

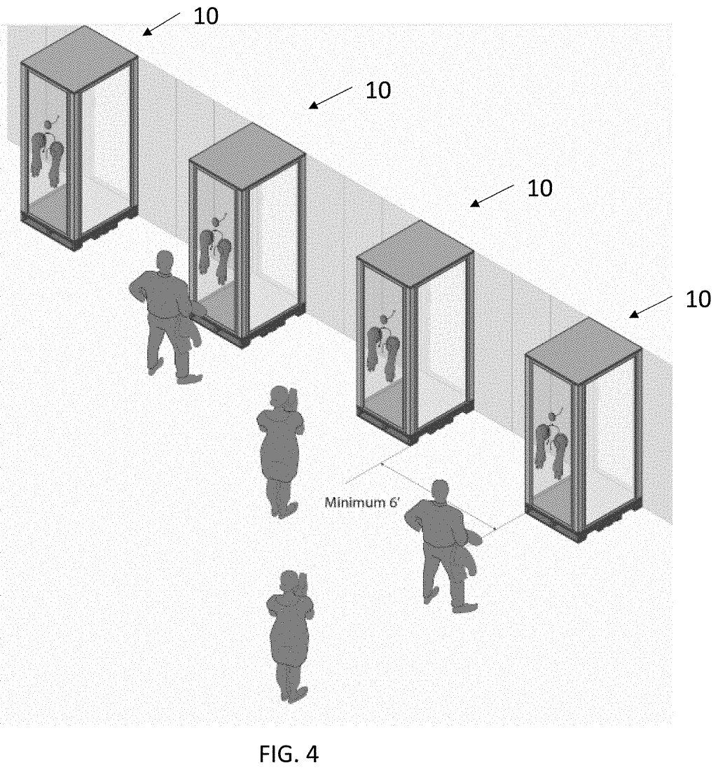

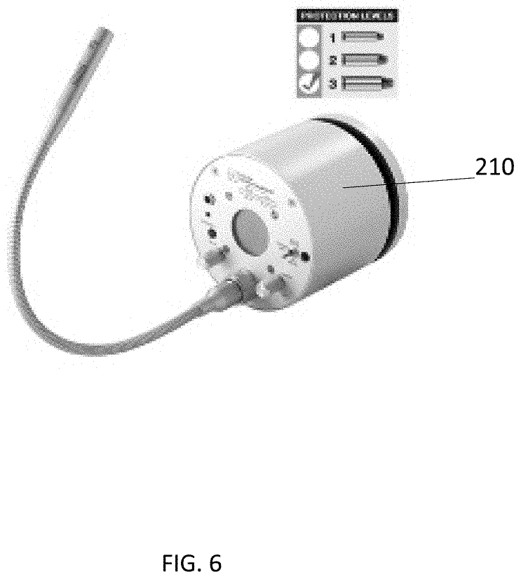

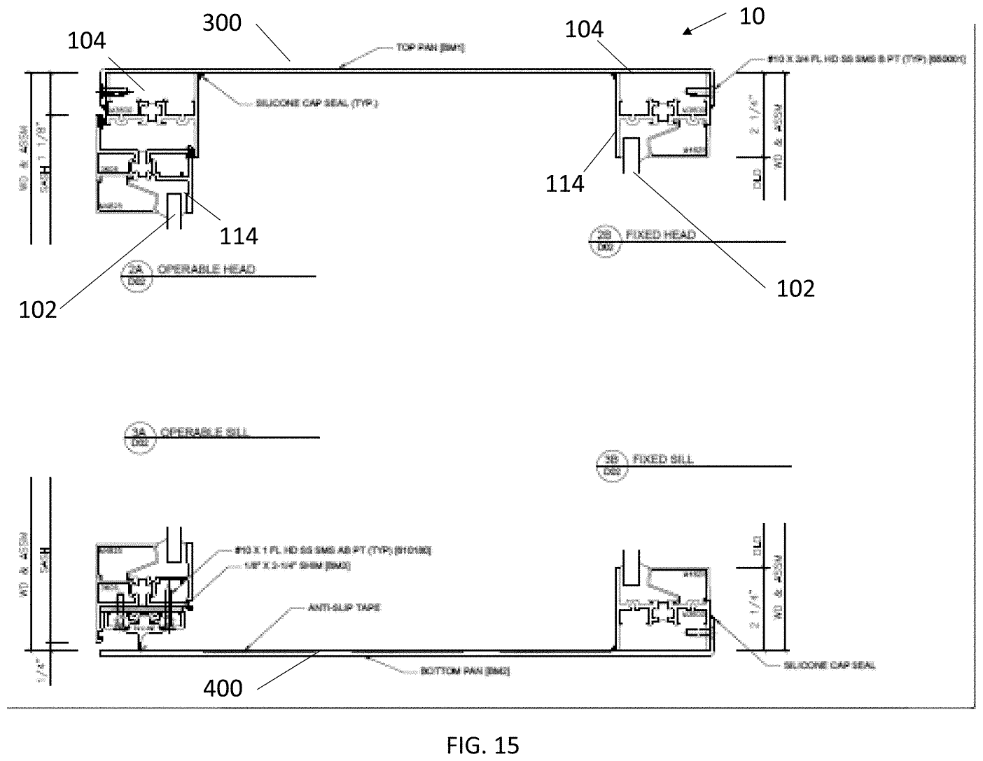

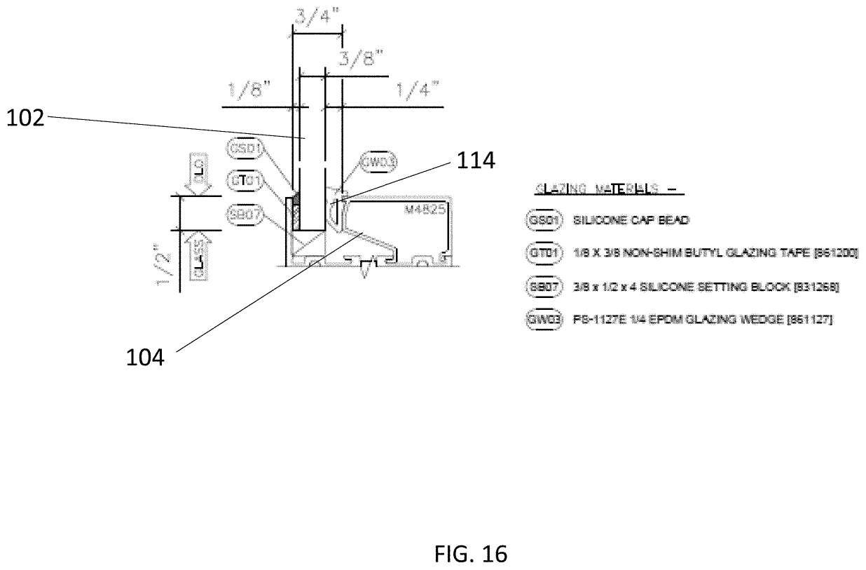

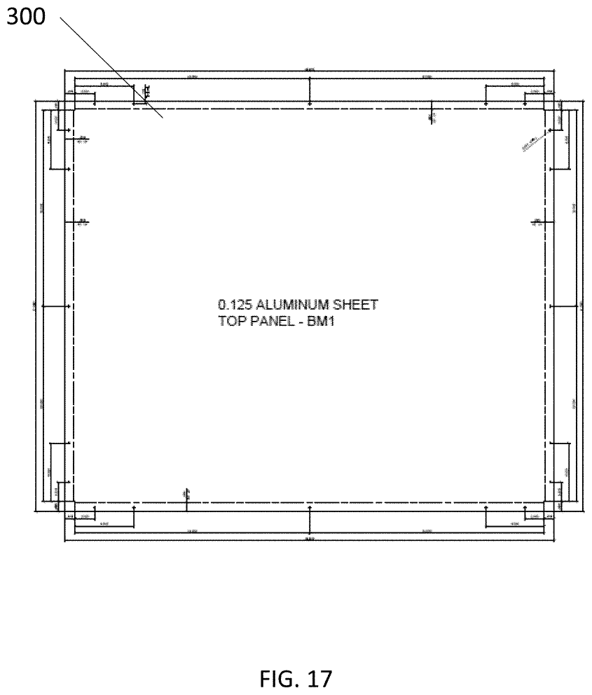

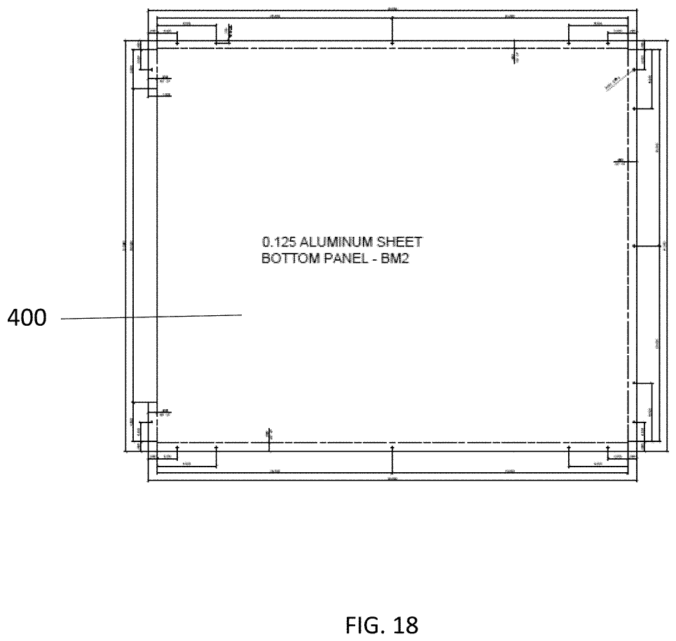

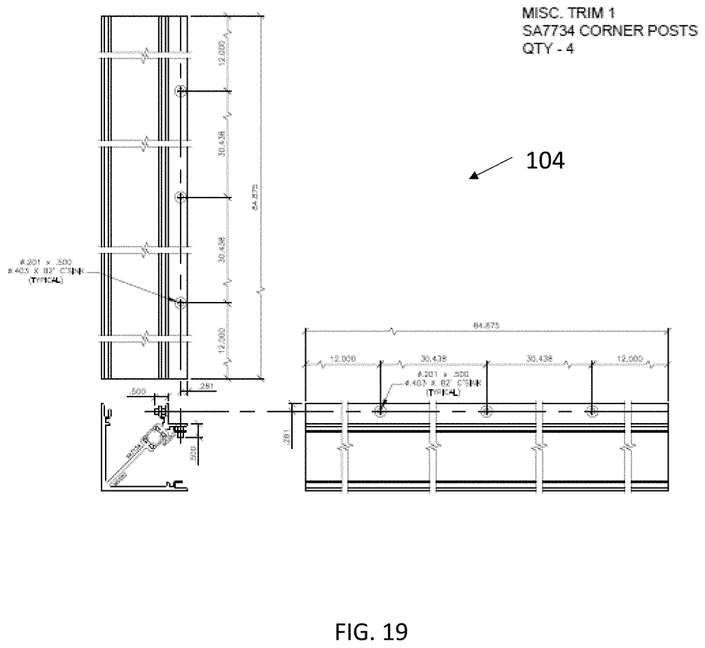

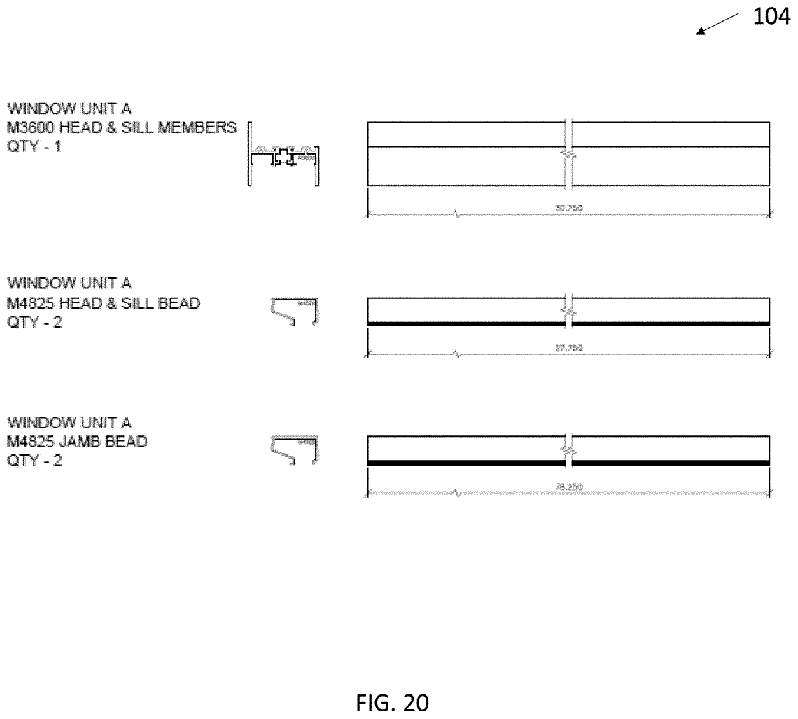

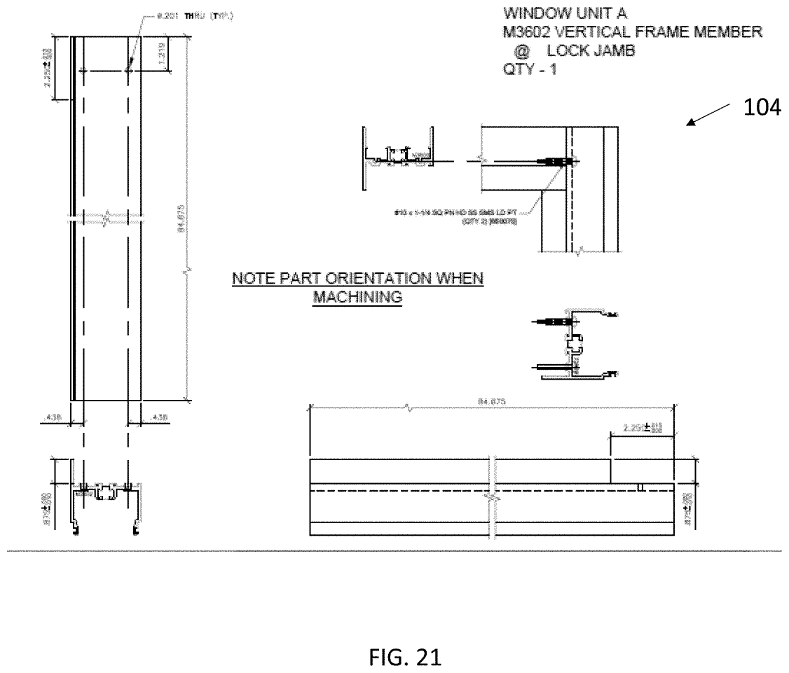

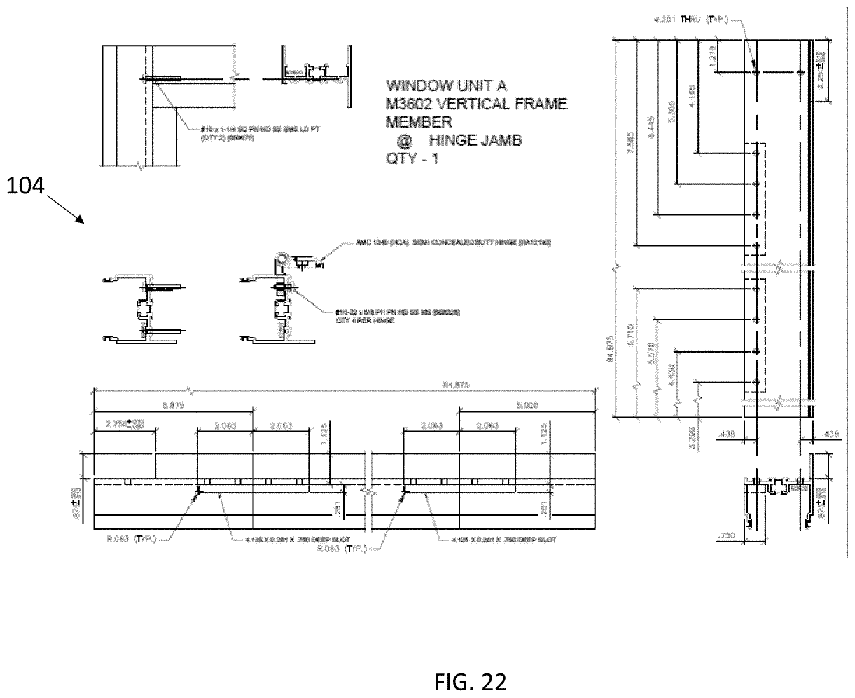

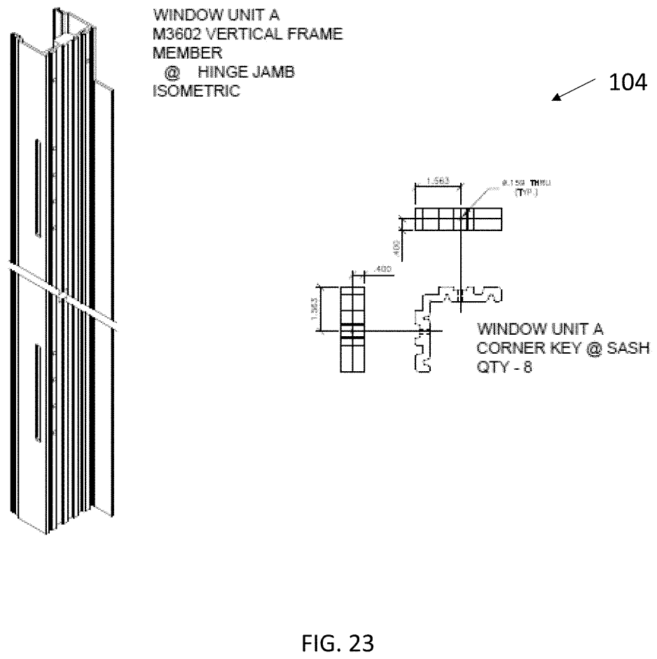

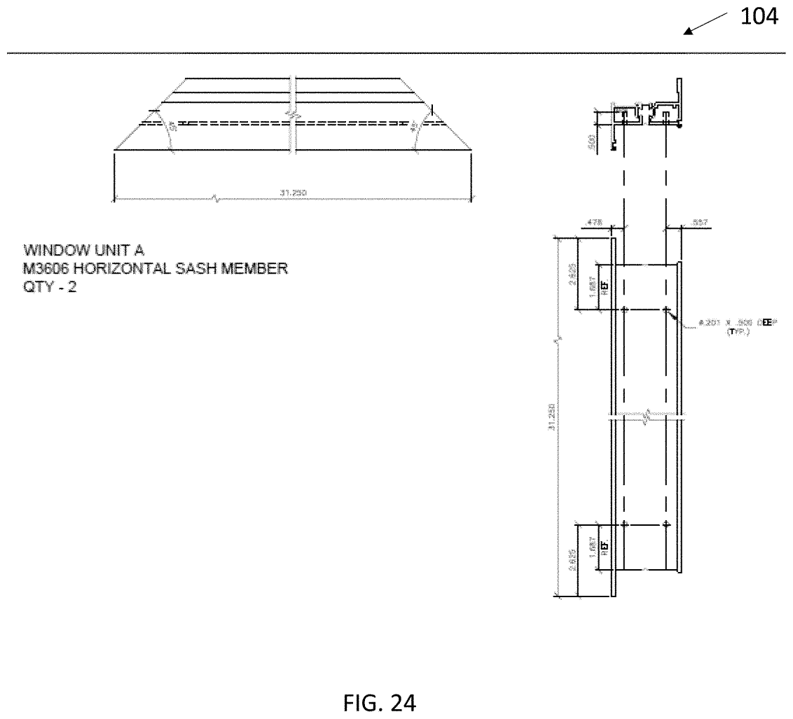

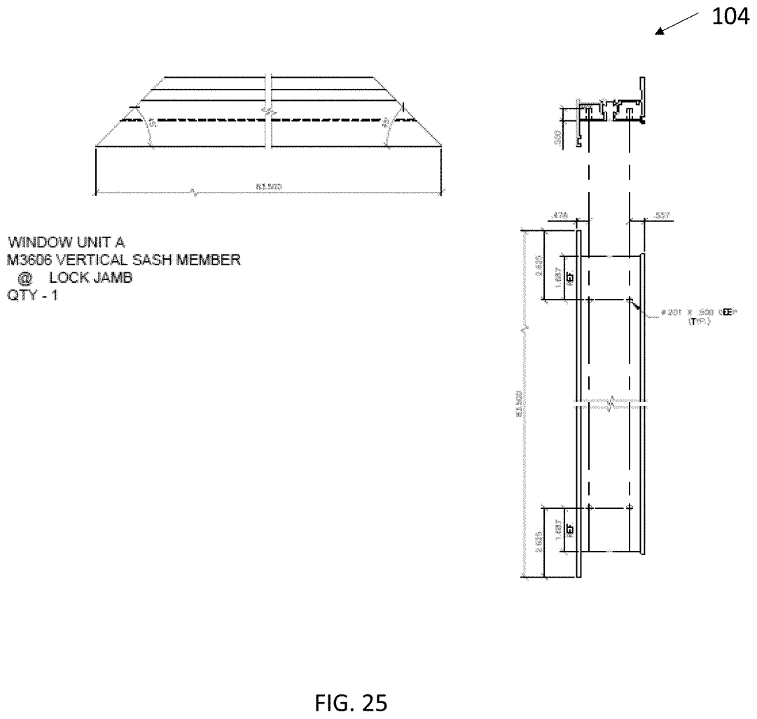

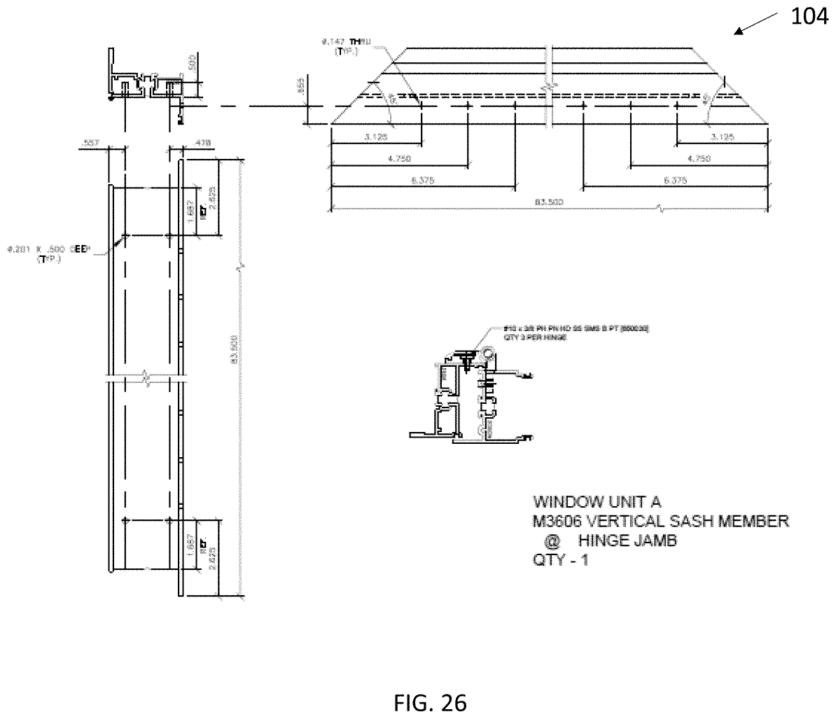

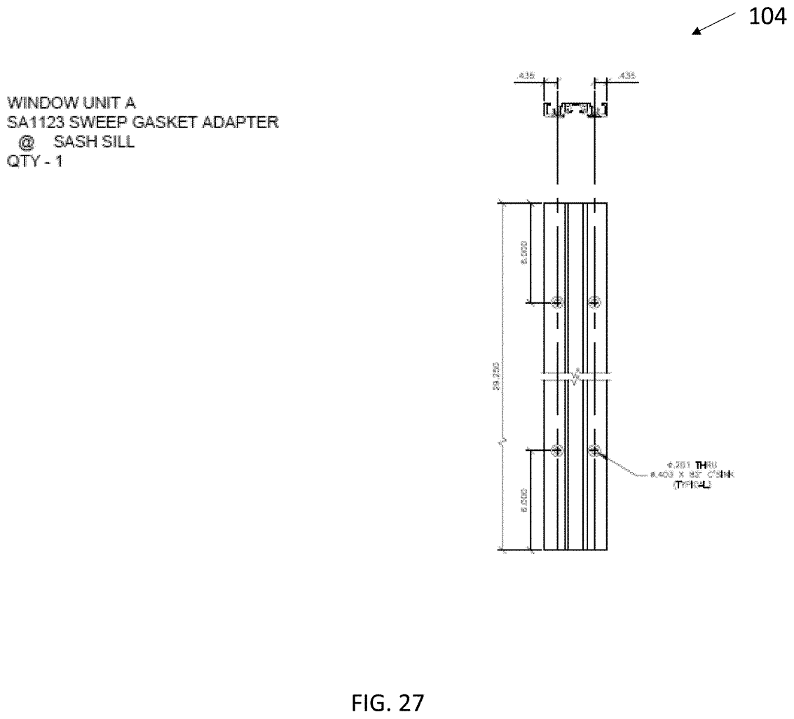

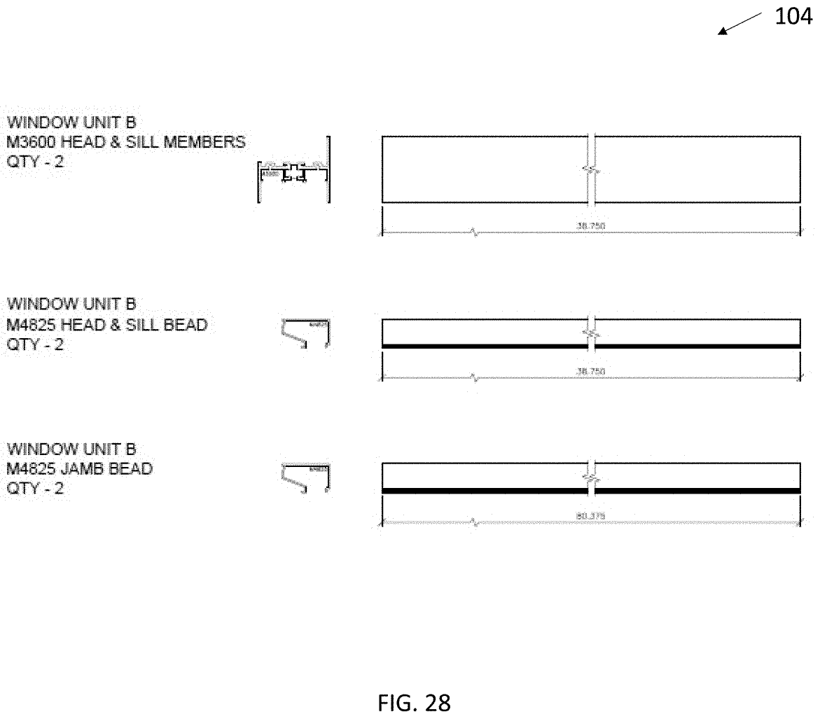

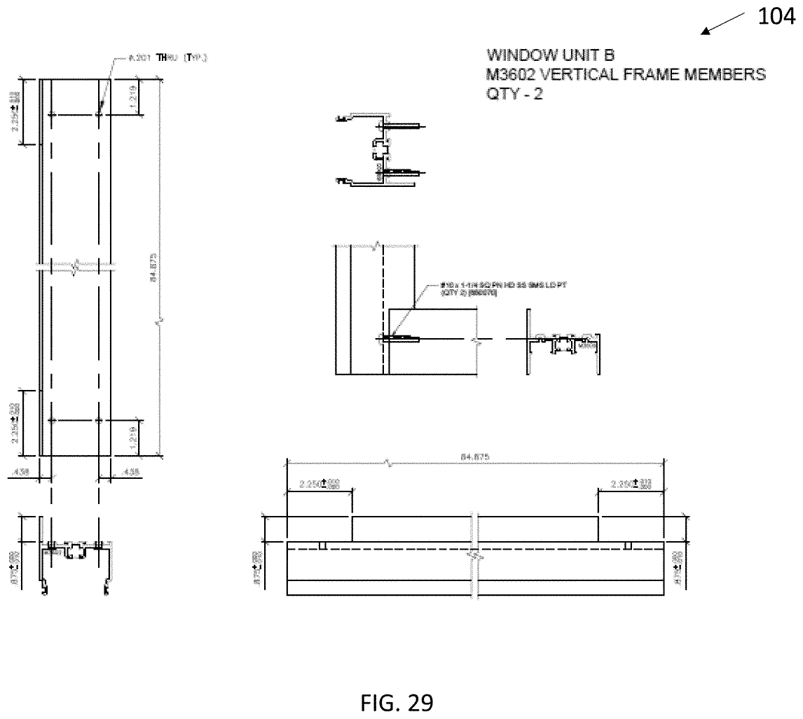

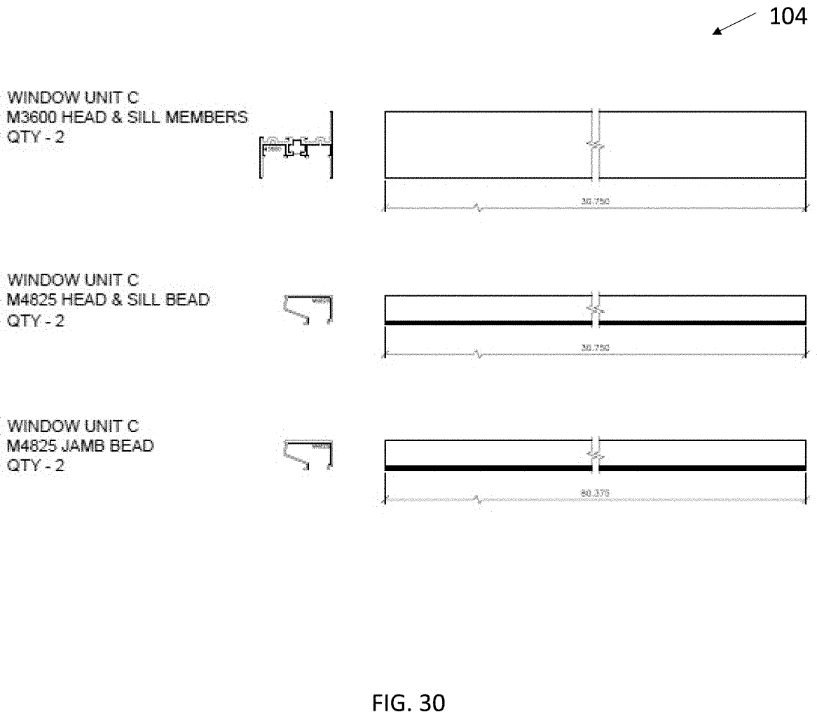

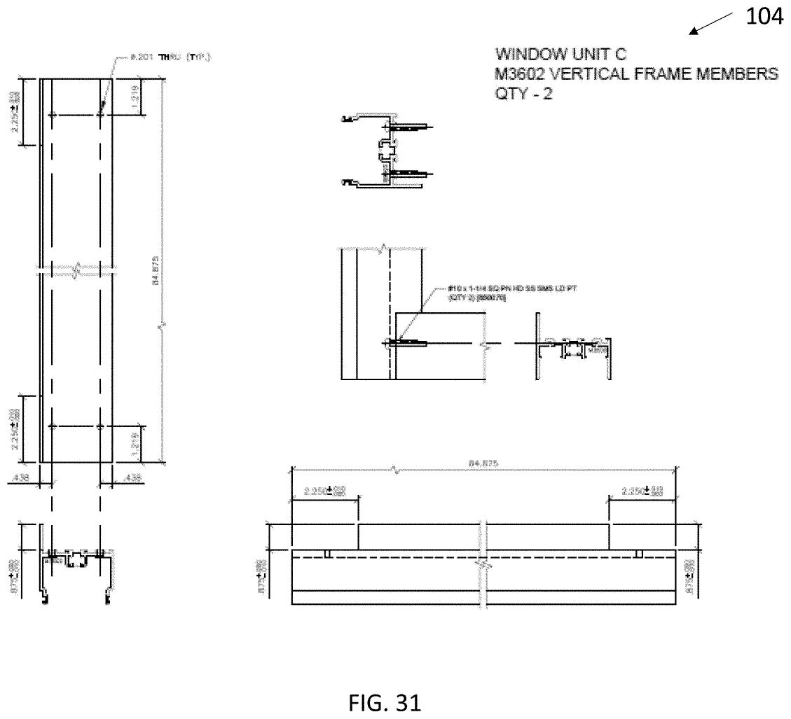

The pathogen examination unit 10, as best illustrated in FIGS. 1-31, is designed to protect healthcare workers from accidental exposure to pathogens while testing or treating patients. In certain embodiments, the healthcare worker is fully encapsulated within a sealed environment. In other embodiments, the healthcare worker is separated from the patient by an impervious surface that at least partially surrounds the healthcare worker.

A. Sealed Unit Construction for Healthcare Workers

According to certain embodiments, as best illustrated in FIGS. 1-32, the pathogen examination unit 10 comprises a working side 12, and entrance side 14, and two non-working sides 16. Each of the sides 12, 14, and 16 may be formed of a frame 101 having one or more frame components 104 that support a panel 102 to form a wall or other similar structure.

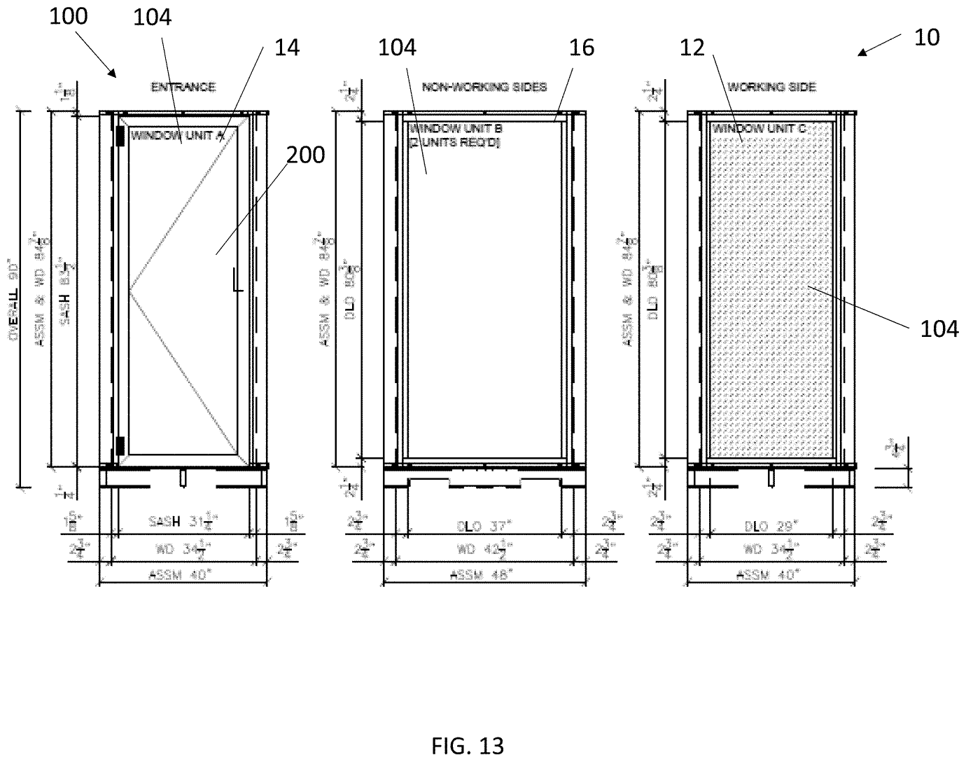

As best illustrated in FIGS. 13-31, the frame component 104 of the frame 101 may be an upper frame component, a lower frame component, a side frame component, etc. In some cases where a plurality of frame components 104 are included, the frame components 104 include the same components; however, in other examples, one frame component 104 may have features that are different from another frame component 104. The frame component 104 may be constructed from various materials including, but not limited to, various metals, woods, plastics, composites, fiber-reinforce polymers, other suitable materials, or combinations thereof. In certain examples, the frame component 104 is formed through an extrusion process, although other techniques may be used to form the frame component 104. In one non-limiting example, the frame component 104 is an extruded aluminum component, but may also be formed of any suitable metal or fiber reinforced polymer, such as carbon fiber. In certain embodiments, the frame component 104 is a unitarily-formed object that is free of joints or other locations that must be sealed, which better ensures a sealed environment within the unit 10. However, a person of ordinary skill in the relevant art would understand that the frame component 104 could be formed of multiple pieces that are joined together so long as the joint between the pieces is fully-sealed and airtight.

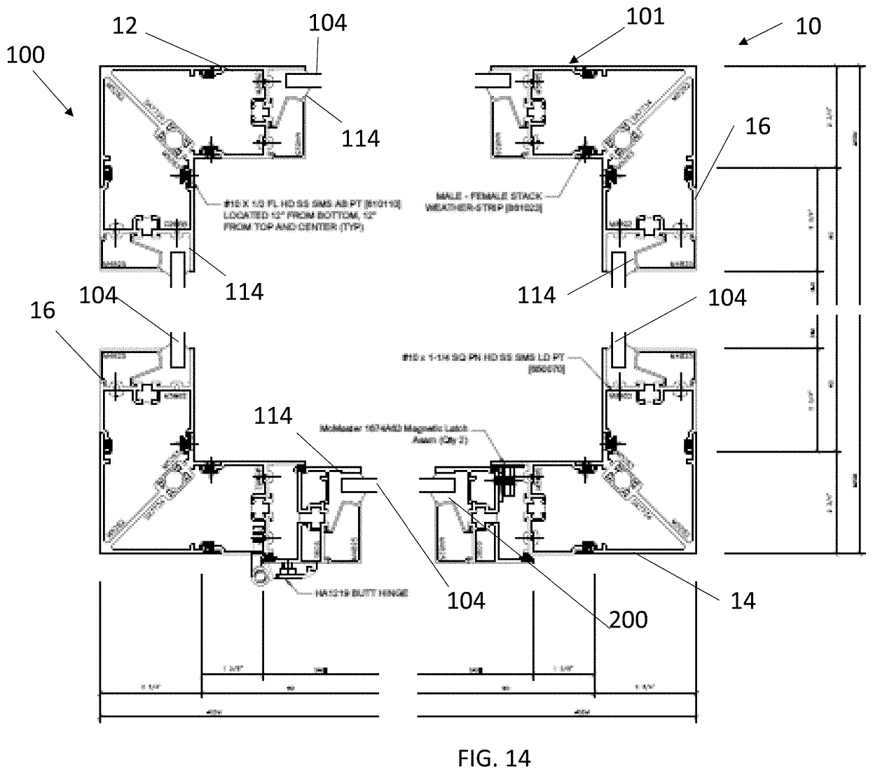

As illustrated in FIGS. 14-16, the frame component 104 of the frame 101 defines at least one channel 114. When the demountable wall system 100 is assembled, at least a portion of the panel 102 is positioned within the channel 114. PCT/US2019/016417 describes the frame components 104 and various methods to join and seal the panels 102 to the frame components in detail, the contents of which is incorporated herein by reference.

At least some of the frame component 104 comprise two channels 114 that are oriented perpendicularly to one another so as to form one of the corners of the unit 10. Each panel 102 is joined to the channel 114 in an airtight manner through the use of gaskets and/or sealant in order to ensure that the joint between the panel 102 and the frame component 104 is fully-sealed and airtight. As a result, when all four sides 12, 14, and 16 comprise the panels 102 that are joined with the frame components 104, the unit is a four-sided enclosure in which the sides and the joints between the sides are fully-sealed and airtight.

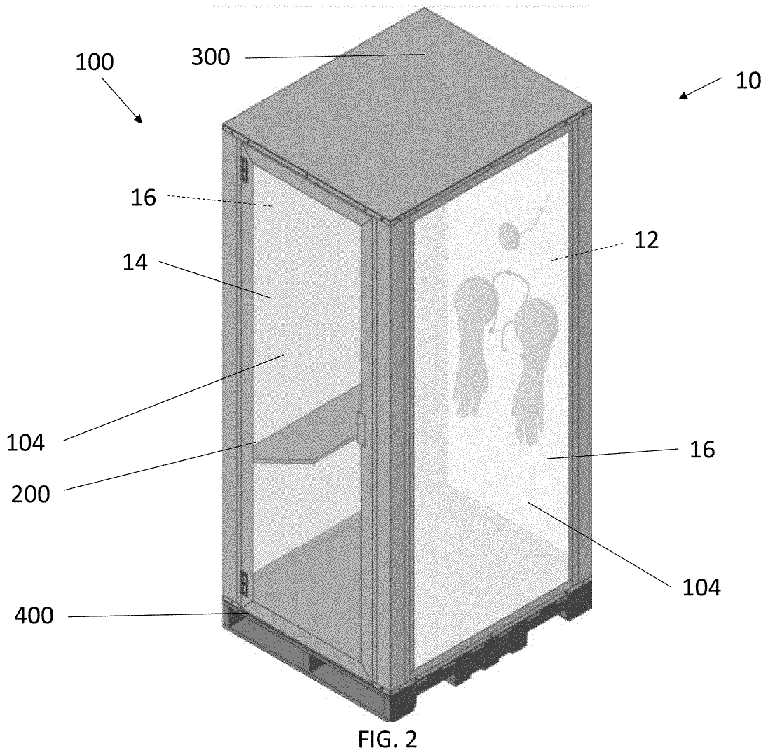

In some embodiments, as best shown in FIGS. 2-3 and 13, the entrance side 14 may further comprise a door 200 that is built into the panel 102 or is attached directly to the frame components 104. The door 200 may be securely held in place and sealed to the unit 10 by use of a magnet and/or gasket that surrounds the edges of the door 200.

The unit 10 may further comprise a top surface 300 and a bottom surface 400, each of which is basically another side formed of the frame 101 having one or more frame components 104 that support the panel 102. In these embodiments, the top surface 300 and the bottom surface 400 are joined to the sides 12, 14, 16 using frame components 104 in the same way that the sides 12, 14, 16 are joined to each other.

The top surface 300 may further comprise a ventilation system 500 that provides a positive pressure inside the unit 10 to further ensure that no air from outside the unit 10 is drawn inside the unit. To achieve the appropriate air pressure inside the unit 10, the air flow could be as little as 50 cubic feet per minute to as much as 300 cubic feet per minute for a 1000 square foot area. In some embodiments, the ventilation system 500 may be powered by a standard outlet.

The dimensions of the unit 10 may range from 2-4 Ft. for the horizontal dimension of each side 12, 14, 16 and may range from 8-10 Ft. for the vertical dimension of each side 12, 14, 16. In certain embodiments, the minimum interior dimensions of 3 Ft.×3 Ft.×7.5 Ft, wherein the width may be determined by the width required by the door 200.

In order for the healthcare worker to interact with the patient, the working side 12 may include a pair of holes 404 to which a pair of industrial gloves 402 are permanently affixed and sealed to the panel 102. The healthcare worker places his or her hands and arms into the permanently-affixed gloves 402 and is able to manipulate the test equipment to collect a specimen or perform a test on the patient.

In some embodiments, as best shown in FIGS. 1, 3, and 6, the working side 12 may further comprise a sealed two-way communication device 210 that allows the healthcare worker inside the unit 10 to communicate with the patient outside the unit 10 without disrupting the sealed environment inside the unit 10.

In certain embodiments, the non-working sides 16 may be opaque to increase patient privacy. In some embodiments, the non-working sides 16 may be configured to provide side panels around the sides of the patient as well. Such a design may be useful for patient privacy or to protect patients from one another when multiple units 10 are positioned adjacent to each other.

B. Unit Assembly and Transport

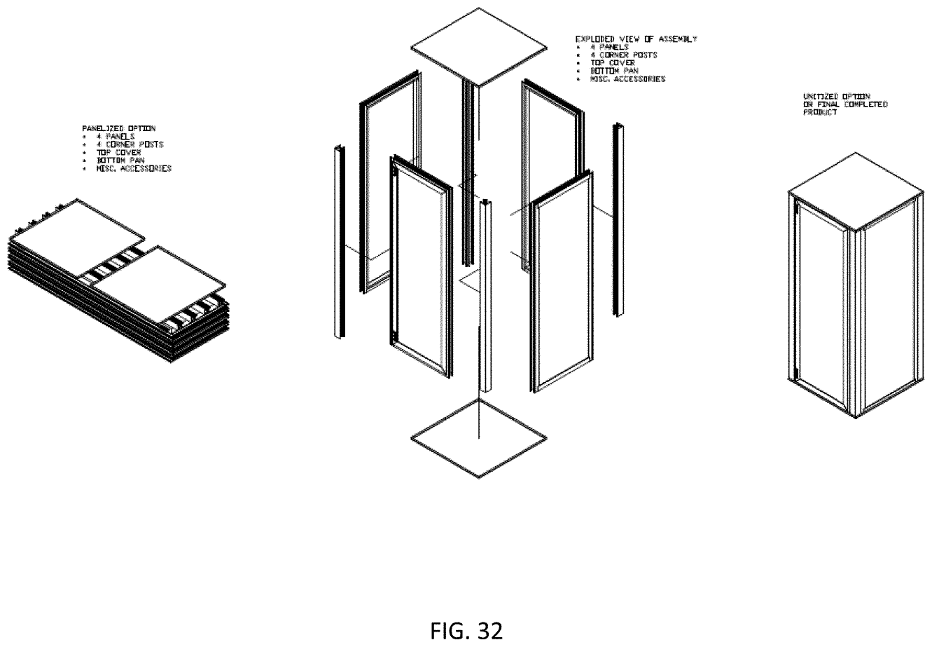

In some embodiments, the unit 10 is designed to be shipped pre-assembled (unitized) when set up must be rapidly executed (or skilled labor is unavailable); or can be shipped panelized (kd) when large volume sales necessitate a more efficient shipping method, as illustrated in FIG. 32. Both approaches are accommodated by the same design and all of the same design components.

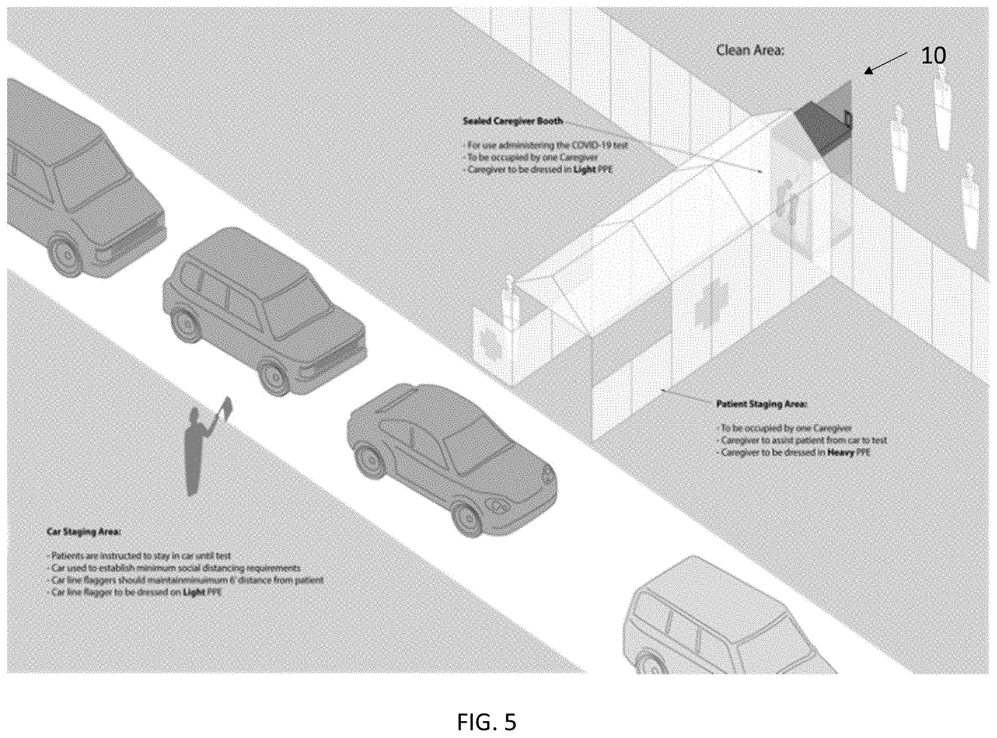

In certain embodiments, the unit 10 may be attached to a pallet, which allows the unit 10 to be easily removed from a flatbed trailer with a forklift or standard pallet jack and moved into position outside a testing facility, in a parking lot, at a drive-through location, or brought inside a facility using a standard pallet jack. An example of a possible use of the unit 10 is illustrated in FIG. 5, which shows patients lined up in their cars and exiting only when it is their turn to approach the unit 10.

In other embodiments, as described above and as best illustrated in FIG. 32, the unit 10 may be panelized where the unit is shipped in a disassembled state and assembled on-site.

Furthermore, the units 10 may be modular in which a plurality of units 10 may be connected to each other with a common non-working side 16 separating each unit and a frame component 104 having three channels for joining the common non-working side 16 to the two top surfaces 300 and the two bottom surfaces 400, respectively. In further embodiments, the panel 102 within each common non-working side 16 may be eliminated to provide a shared open space for the healthcare workers within the joined units 10. Such a design may also reduce the costs associated with providing a separate ventilation system 500 for each unit 10.

C. Test Kit or Specimen Collection

The working side 12 may further comprise a means of collecting the specimen or used test kit from the patient without creating exposure to the healthcare worker inside the unit 10.

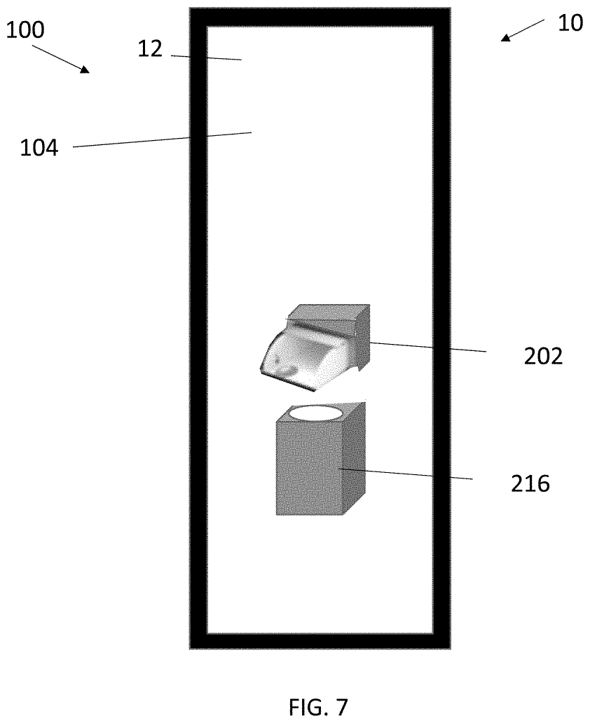

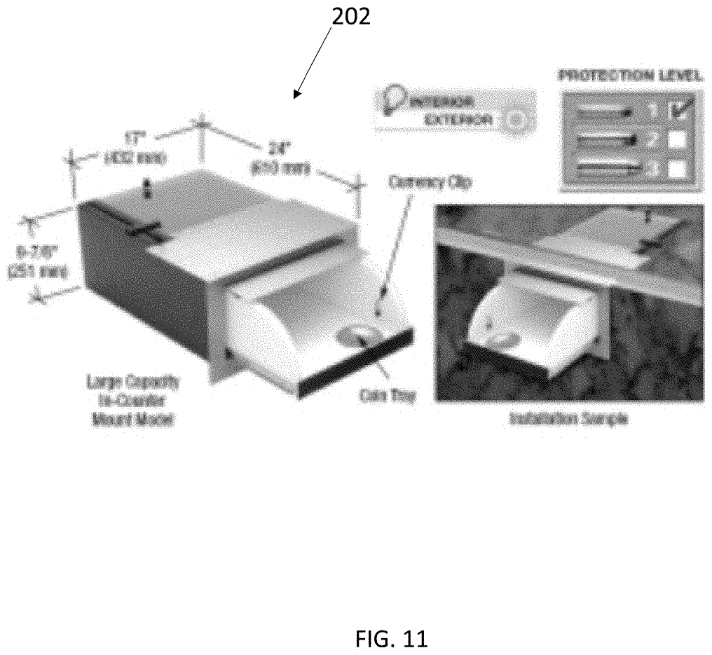

For example, as shown in FIGS. 7 and 11, a collection drawer 202 may be attached to the working side 12. From within the unit 10, the healthcare worker may manipulate the drawer 202 to open out so that the patient can place the specimen or test kit inside the drawer 202 (similar to the mechanism seen at banks and pharmacy drive-through windows). But, instead of pulling the specimen or used test kit into the unit 10 like a bank teller or pharmacist might do, the healthcare worker may instead manipulate the drawer 202 to open at the bottom to dispose of the test kit or place the specimen into an area that is sealed off from the inside of the unit 10 for later collection by appropriate personnel.

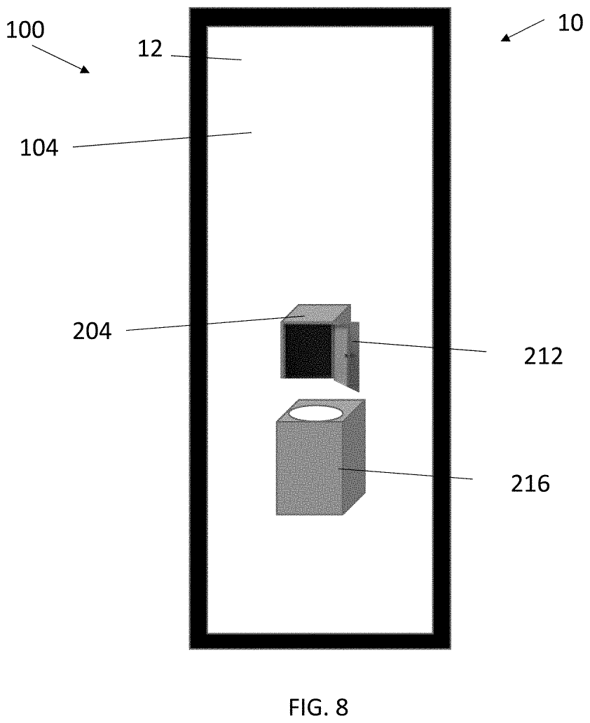

In further embodiments, as shown in FIG. 8, a collection bin 204 may be attached to the working side 12. The collection bin 204 may have a door 212 can be opened by the healthcare worker from within the unit 10. The patient places the used test kit or specimen into the open collection bin 204 and closes the door. The appropriate personnel can then access the collection bin 204 to retrieve the specimen or test kit from the same door 212 or from another door on a side of the collection bin that is not exposed to the patient. In other embodiments, like the drawer concept, the healthcare worker may be able manipulate the collection bin 204 from within the unit 10 to open at the bottom to dispose of the test kit or place the specimen into an area that is sealed off from the inside of the unit 10 for later collection by appropriate personnel.

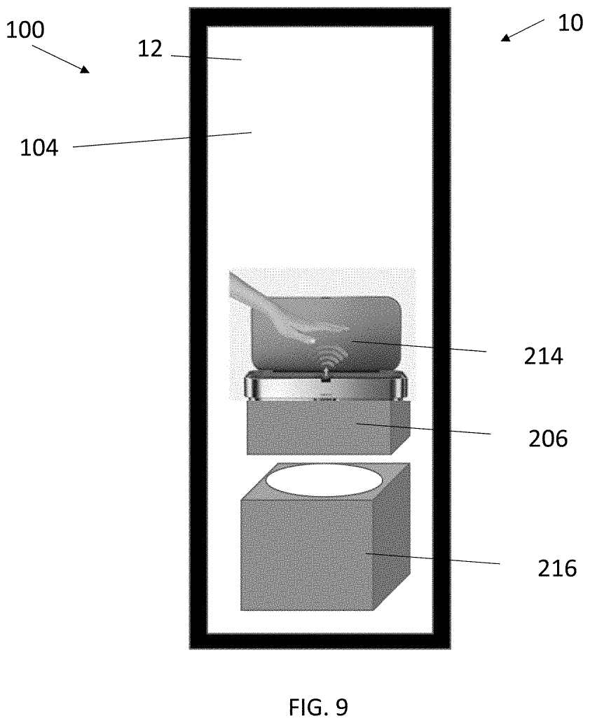

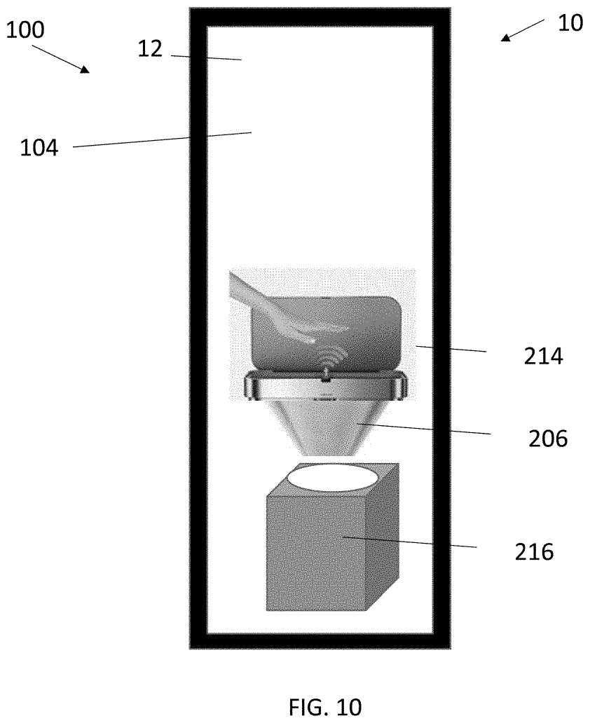

In further embodiments, as shown in FIGS. 9-10, a receptacle or funnel 206 may be attached to the working side 12 with a flap 214 that is held closed by a spring or other device. Alternatively, the flap 214 may be opened by waving a hand over the flap 214 or otherwise using touchless technology to open the flap 214. When the flap 214 opens, the patient places the used test kit or specimen through the opening below the flap 214 and into the receptacle or funnel 206 to allow the used test kit or specimen to drop into an area that is sealed off from the inside of the unit 10 for later collection by appropriate personnel.

In some embodiments, as shown in FIGS. 7-10, a container 216 may join to the bottom of the drawer 202, the collection bin 204, or the receptacle or funnel 206 to allow the specimen or used test kit to drop directly into the container 216 which is then sealed when the bottom of the drawer closes. The sealing device may comprise of a lid that joins with the container 216 (similar to a Tupperware® container) or a mechanism that seals the sides of the container 216 around the specimen or used test kit at the top (similar to a mechanism that seals a potato chip bag after it is filled with chips or to a mechanism that seals a container after a soiled diaper has been placed inside). In this manner, the sealed container 216 is now safe to handle by healthcare workers.

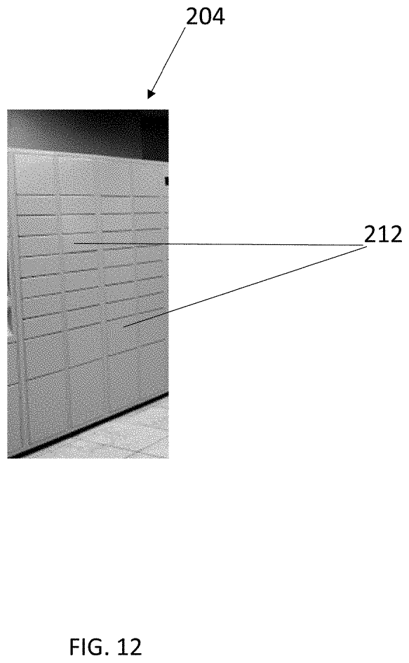

In some embodiments, as shown in FIG. 12, the patient may be provided a code that will open a door 212 to a collection bin that is part of a bank of collection bins or the healthcare worker may be provided with a means to open the door 212 from within the unit 10. Rather than placing the test kit into a drawer attached to the unit 10, the patient places the used test kit or specimen into the open collection bin 204 and closes the door 212. The appropriate personnel can then access the collection bin 204 to retrieve the specimen or test kit from the same door or from another door on the opposite side of the collection bin 204.

D. Testing Procedure

In certain embodiments, a patient approaches the working side 12, either from a car or through a tent structure placed outside of the hospital or other facility. The patient may be placed inside of the unit 10 or outside of the unit 10. The patient and the healthcare worker confer about the procedure to be performed through an appropriate two-way communication device 210.

The healthcare worker then administers the test or specimen collection, utilizing the gloves 402 that are attached to the working side 12. For example, for COVID-19 testing, the healthcare worker removes a test stick from packaging and inserts the test stick into the nose of the patient (typically inducing a sneeze from the patient). The caregiver places the test stick into a sealed tube for testing. The patient then leaves the unit.

In some instances in which the test kit or specimen collection units are not included with the unit 10, a healthcare worker enters the patient area and collects the sealed tube after the patient leaves. The healthcare worker wipes down the tube exterior and places it into a sealed bag. The healthcare worker then removes the sealed bag for testing.

In some instances where the patient area of the unit 10 must be cleaned manually, a healthcare worker enters the patient area and cleans all surfaces and any items that are located within the patient area of the unit 10. The surfaces and items may be cleaned by fogging or by applying a liquid disinfectant spray, wiping down the surfaces and items, and replacing the plastic liners on the gloves 402.

E. Non-Sealed Unit Construction

In other embodiments, the entrance side 14 may not include a door and may be open for the healthcare worker to pass into and out of the unit 10. These embodiments may be suitable for cases where airborne exposure routes are not a concern (e.g., where the purpose of the unit 10 is to protect the healthcare worker from direct exposure to pathogens that are transmitted by the patient through coughing, sneezing, or other human contact, and the relevant pathogen is not communicable through the air or is otherwise sufficiently unlikely to reach the healthcare worker's side of the unit 10.) In the non-sealed embodiments, the unit 10 may or may not include the non-working sides 16. In other words, the unit 10 may solely comprise a paneled working side 12 and/or may also comprise paneled non-working sides 16 and/or may also comprise the top surface 300 and/or may also comprise the bottom surface 400.

F. Sealed Unit Construction for Patients

In certain embodiments, it may be desirable to place the patient inside the unit 10 and have the healthcare workers treat the patient from outside of the unit 10. In these embodiments, the sides 12, 14, 16, top surface 300, and bottom surface 400 would continue to be assembled as described above. Modifications may include reversing the side from which the gloves 402 are affixed to the working side 12 so that the permanently-affixed gloves 402 are directed into the unit 10. Additional modifications may include using a negative pressure inside the unit 10 so that pathogens will not pass out of the unit 10, and adding an appropriate filter to the ventilation system 500 of the unit 10 that is suitable for trapping pathogens so that they are not released into the atmosphere outside the unit 10. The CDC recommends that patients with known or suspected COVID-19 should be placed in an airborne infection isolation room (“AIIR”), if available. AIIR's Qualification for new structures is 12 ACH and should be pulled through an appropriate filter so as to negate further airborne distribution of the pathogens.

G. Panel Material & Treatment

The panel 102 may be formed of any suitable surface that can be treated or cleaned efficiently to provide a sanitary area for patients to be tested/treated, including but not limited to glass, plastics, metals, and other porous or non-porous surfaces.

Surface characteristics of the panel 102 may include but are not limited to the following:
Easy to maintain, repair and clean
Does not support microbial growth
Non porous and smooth
Has acoustic properties (e.g. sound absorption), where applicable
Inflammable-Class 1 fire rating, low smoke toxicity
Durable
Sustainable
Low-VOC (no off-gassing)
Cost-effective (initial and life-cycle cost-effectiveness)
Slip-resistant (appropriate coefficient of friction)
Easy to install, demolish, and replace
Has compatible substrate and materials for surface assemblies
Seamless
Resilient, impact-resistant
Control reflectivity/glare
Has options for color, pattern, and texture
Made of non-toxic/non-allergenic materials

For example, the panel 102 may contain or be otherwise treated with antimicrobial agents including but not limited to the following:
Silver ions that may be diffused into the upper layers of the panel 102 so that the silver ions may interact with bacteria or other pathogens and destroy them by disabling their metabolism and disrupting their division mechanism. The silver ions may also create a reflective surface on the panel 102 that gives the panel 102 a prismatic effect that improves the disinfecting capabilities of certain light treatments that are described more fully in the next section.
Silicon oxide.
Copper ions.
Ultraviolet-active titanium dioxide, such that when the panel 102 is treated with UV light, photocatalysis occurs that chemically breaks down organic contaminants on the surface of the panel 102.
Visible light-active titanium dioxide, such that when the panel 102 is treated with visible light, photocatalysis occurs that chemically breaks down organic contaminants on the surface of the panel 102.
LIQUID GUARD®, which creates an invisible physical barrier on the panel 102 to destroy microbes.
Kastus® Glass, which creates a super-hydrophilic surface on the panel 102. The technology reacts with moisture in the air to form reactive oxygen species that constantly attack and destroy harmful bacteria that they encounter.
Other antimicrobial films or tents that can be applied to the surface of the panel 102 (similar to methods used to apply window tinting and the like).
Any of the above treatment technologies or other technologies can include nano coatings, where the treatment is applied at the nano level to allow the particles to better penetrate the surface of the panel 102.

H. Unit Cleaning

Additionally and/or alternatively to the treatment options described above, the panel 102 may be sanitized between patients via any suitable cleaning method that conforms to CDC guidelines for surface disinfection of healthcare equipment that contact intact skin (such as blood pressure cuffs and stethoscopes). These guidelines state that noncritical medical equipment surfaces should be disinfected with an EPA-registered low- or intermediate-level disinfectant.

For situations where the panels 102 are repeatedly disinfected between patients, the use of glass as the panel material may have certain advantages over epoxy or other plastic materials that may begin to breakdown or become hazy after repeated cleaning. Glass will maintain a clear surface even after hundreds or even thousands of cleanings, thus providing the patient and the healthcare worker with better visibility of each other so as to facilitate communication and make the interaction between the healthcare worker and the patient more personal and less stressful. Furthermore, the clean and fresh appearance of glass panels 102 may provide a greater sense of confidence for patients that the unit 10 is clean and sanitary.

In addition to cleaning the panel 102, the permanently-affixed gloves 406 may be covered by a pair of disposable gloves that are removed after each patient leaves the unit 10 and replaced by a fresh pair of disposable gloves.

Additional cleaning methods may include various light treatments. For example, ultraviolet germicidal irradiation (“UVGI”) is a disinfection method that uses short-wavelength ultraviolet (“UV-C”) light to kill or inactivate microorganisms by destroying nucleic acids and disrupting their DNA, leaving them unable to perform vital cellular functions. UVGI has been successfully used to treat solid surfaces, such as the interior surfaces of an ambulance. While the UV irradiance varied considerably within the ambulance based on the variety of surface locations (16 seconds for the most irradiated locations to 15 hours for the least irradiated locations), the technology would be able to irradiate the flat working side 12 in a time period that is much closer to 16 seconds. Furthermore, the UVGI light fixture can be mounted inside the unit 10 and directed through the panel 102. As a result, a healthcare worker does not need to clean the working surface 12 between patients, other than to replace the disposable gloves on the permanently-affixed gloves 402.

In embodiments where the UVGI fixture is positioned outside of the unit 10, the addition of reflective particles within the panel 102 will create a reflective surface with a prismatic effect that further increases the efficacy of the UVGI treatment. Installing multiple UVGI light fixtures and/or having a track of UVGI light fixtures that moves across the surface of the panel 102 will also improve the efficacy of the UVGI treatment. The movement of the UVGI light fixture along the track may or may not be motorized to move at a specified rate and number of passes sufficient to attain the level of disinfection required.

Light sources that can provide the desired UVGI treatment are ultraviolet light-emitting diodes (“UV-C LED”) lamps that emit UV light at selectable wavelengths within the 200-300 nm range. Additional light sources that may also provide suitable treatment include but are not limited to pulsed xenon lamps (which emit UV light across the entire UV spectrum with a peak emission near 230 nm) and mercury lamps (which operate at low vapor pressure and emit UV light at the 253.7 nm line).

In other embodiments, a plurality of lasers may be used to disinfect the panel 102 by directing the lasers over the entire surface of the panel 102. A laser directs pulses of light at the surface of the panel 102. As the beam heats up the dirty surface, the dirt (top layer) either evaporates or peels off. The material underneath does not absorb the light, thus staying cold and unaffected. Depending on the strength of the laser, it can fire a few strong pulses that can clean a larger area per pulse or it can emit hundreds of thousands of weaker pulses, all of which touch the surface and achieve the cleaning effect. Since ultraviolet radiation for lasers may be designed within the 200 nm to 300 nm band necessary to achieve UVGI, lasers may be particularly effective in eliminating microbes through both UVGI and thermal means.

In further embodiments, the panel 102 may be heated to disinfect the surface. For example, the panel 102 may contain a grid made of metal and resin that is attached to the glass or imbedded within the glass. When activated, the resistance in the metal generates enough heat raise the temperature of the panel 102 to the recommended temperature and for the recommended time to achieve sterilization. In some cases, the recommended amount is at least 170 degrees F. for at least 30 seconds, but may be more or less depending on the circumstances and the particular need.

As other examples, the panel 102 may be steam sterilized by applying a blast of steam on the surface of the panel 102 for the recommended time. In some cases, the recommended amount is at least 132 degrees F. for at least 3 minutes, but may be more or less depending on the circumstances and the particular need.

In yet more embodiments, the panel 102 may be disinfected using jets or sprinklers that cover the surface of the panel 102 and the permanently-affixed gloves 402 with a chemical disinfectant. The disinfectant may remain on the surface or be rinsed off as needed. Suitable examples of chemical disinfectants include but are not limited to alcohol, chlorine, glutaraldehyde, p-Chloro-o-benzylphenol, o-Phenylphenol, hydrogen peroxide, idophors, ortho-phthaladehyde (“OPA”), peracetic acid.

In still further embodiments, the panel 102 may be disinfected using cryochamber disinfectant technology.

The above cleaning methods may be applied alone or in combination with each other or with one or more of the treatment methods to achieve a cleaning time between patients of less than 30 seconds, preferably less than 20 seconds, and more preferably less than 10 seconds.

Each of the above cleaning methods may similarly be employed to disinfect the inside surfaces and items within the unit 10 as well as the exterior surfaces of the unit 10.

I. Possible Compliance with OSHA Standards

In some cases, the unit 10 may be characterized as a portable workspace, which may comply with certain OSHA standards, including but not limited to (1) engineering controls to prevent “atmospheric contamination” under 29 C.F.R. § 1910.134(a)(1), and (2) “personal protective equipment” under 29 C.F.R. § 1910.132 and § 1910.138. See § 1910.134(a)(1) (“In the control of those occupational diseases caused by breathing air contaminated with harmful dusts, fogs, fumes, mists, gases, smokes, sprays, or vapors, the primary objective shall be to prevent atmospheric contamination. This shall be accomplished as far as feasible by accepted engineering control measures (for example, enclosure or confinement of the operation, general and local ventilation, and substitution of less toxic materials).”); § 1910.132(a) (“Protective equipment, including personal protective equipment for eyes, face, head, and extremities, protective clothing, respiratory devices, and protective shields and barriers, shall be provided, used, and maintained in a sanitary and reliable condition wherever it is necessary by reason of hazards of processes or environment, chemical hazards, radiological hazards, or mechanical irritants encountered in a manner capable of causing injury or impairment in the function of any part of the body through absorption, inhalation or physical contact.”); § 1910.138 (“Employers shall select and require employees to use appropriate hand protection when employees' hands are exposed to hazards such as those from skin absorption of harmful substances; severe cuts or lacerations; severe abrasions; punctures; chemical burns; thermal burns; and harmful temperature extremes.”).

As one OSHA Standard Interpretation states, “[i]n order for a device or method to qualify as an engineering control for an airborne substance, it must reduce the amount of the airborne substance in the surroundings in which individuals move about. For that matter, the ultimate objective of an engineering control is to improve the environment to the point that personal protective equipment need not be worn.” OSHA Interpretive Letter (Aug. 13, 1981).

J. Benefits

In certain cases, the unit 10 provides a mobile specimen collection PPE that can be easily mobilized by governments for rapid scale up and quick deployment to any site to meet testing demand. The unit 10 allows for pandemic response for specimen collection, improving preparedness and response efforts.

In some embodiments, the unit 10 reduces PPE costs (i.e. powered air-purifying respirators (“PAPR”), hazmat suit, etc.) and demand by enclosing medical personnel in a positive pressurized product where they do not need to wear disposable isolation gowns, N95 respirator and a face shield/goggles and gloves.

By reducing the cleaning time to 30 seconds or less (and assuming that the patient testing time remains unchanged at approximately 6-7 minutes per patient), the whole process now takes about 6.5 minutes. The unit 10 may increase specimen collection capacity to 9 patients per hour over traditional testing of 2 patients per hour, thereby quickly meeting specimen collection and testing demand.

The unitized (pre-assembled) units can be assembled in under 20 minutes, and panelized units can be assembled in less than 1 hour to speed deployment. Units can also be disassembled easily and quickly by unskilled labor to move units to new specimen collection sites, if needed. The units 10 minimize waste, as the units 10 are easily cleaned, taken apart, and stored for future use.

A collection of exemplary embodiments, including at least some explicitly enumerated as “ECs” (Example Combinations), providing additional description of a variety of embodiment types in accordance with the concepts described herein are provided below. These examples are not meant to be mutually exclusive, exhaustive, or restrictive; and the invention is not limited to these example embodiments but rather encompasses all possible modifications and variations within the scope of the issued claims and their equivalents.

EC 1. A pathogen examination unit comprising: a working side, two non-working sides, and an entrance side, wherein the working side comprises a panel that is positioned between a healthcare worker and a patient.

EC 2. The pathogen examination unit of any of the preceding or subsequent example combinations, wherein the panel comprises an antimicrobial treatment.

EC 3. The pathogen examination unit of any of the preceding or subsequent example combinations, wherein the antimicrobial treatment comprises at least one of silver ions, silicon oxide, copper ions, ultraviolet-active titanium dioxide, visible light-active titanium dioxide, LIQUID GUARD®, Kastus® Glass, or other antimicrobial films or tents.

EC 4. The pathogen examination unit of any of the preceding or subsequent example combinations, wherein the panel is formed of glass.

EC 5. The pathogen examination unit of any of the preceding or subsequent example combinations, wherein the entrance side comprises a panel comprising a door, wherein the door forms an airtight seal through use of at least one of a magnet or a gasket.

EC 6. The pathogen examination unit of any of the preceding or subsequent example combinations, wherein each non-working side comprises a panel.

EC 7. The pathogen examination unit of any of the preceding or subsequent example combinations, further comprising an upper surface and a lower surface, wherein the working side, two non-working sides, the entrance side, the upper surface, and the lower surface are joined to each other via frame components.

EC 8. The pathogen examination unit of any of the preceding or subsequent example combinations, wherein each frame component comprises two channels into which an edge of two panels meeting at a perpendicular angle are inserted.

EC 9. The pathogen examination unit of any of the preceding or subsequent example combinations, wherein each frame component is formed of unitary construction.

EC 10. The pathogen examination unit of any of the preceding or subsequent example combinations, wherein the pathogen examination unit is sealed when the door is closed.

EC 11. The pathogen examination unit of any of the preceding or subsequent example combinations, further comprising a ventilation system connected to the pathogen examination unit.

EC 12. The pathogen examination unit of any of the preceding or subsequent example combinations, wherein the ventilation system is configured to supply a positive air pressure inside the unit when a healthcare worker is located inside the unit.

EC 13. The pathogen examination unit of any of the preceding or subsequent example combinations, wherein the ventilation system is configured to supply a negative air pressure inside the pathogen examination unit when a patient is located inside the unit.

EC 14. The pathogen examination unit of any of the preceding or subsequent example combinations, wherein the ventilation system is configured with an additional filtration means for removing pathogens from exhausted air when a patient is located inside the pathogen examination unit.

EC 15. The pathogen examination unit of any of the preceding or subsequent example combinations, wherein the panel is cleaned by application of at least one of light, heat, steam, chemicals, and cold.

EC 16. The pathogen examination unit of any of the preceding or subsequent example combinations, wherein the application of light further comprises application of ultraviolet germicidal irradiation for less than 30 seconds, preferably less than 20 seconds, and more preferably less than 10 seconds.

EC 17. The pathogen examination unit of any of the preceding or subsequent example combinations, wherein a light source that provides the ultraviolet germicidal irradiation is positioned within the pathogen examination unit and directed through the panel.

EC 18. The pathogen examination unit of any of the preceding or subsequent example combinations, wherein a light source that provides the ultraviolet germicidal irradiation is positioned outside the pathogen examination unit and directed onto the panel.

EC 19. The pathogen examination unit of any of the preceding or subsequent example combinations, wherein the panel comprises prismatic properties that assist with reflecting light by the light source to achieve greater irradiation.

EC 20. The pathogen examination unit of any of the preceding or subsequent example combinations, wherein a light source that provides the ultraviolet germicidal irradiation provides ultraviolet light within a 200-300 nm range.

EC 21. The pathogen examination unit of any of the preceding or subsequent example combinations, wherein the light source is at least one of ultraviolet light-emitting diodes, pulsed xenon lamps, and mercury lamps.

EC 22. The pathogen examination unit of any of the preceding or subsequent example combinations, wherein a light source that provides the ultraviolet germicidal irradiation travels across a surface of the panel.

EC 23. The pathogen examination unit of any of the preceding or subsequent example combinations, wherein the application of light further comprises application of lasers for less than 30 seconds, preferably less than 20 seconds, and more preferably less than 10 seconds.

EC 24. The pathogen examination unit of any of the preceding or subsequent example combinations, wherein the application of heat further comprises use of a metallic grid attached to or imbedded within the panel to generate heat and raise a panel temperature to a level sufficient to disinfect the panel.

EC 25. The pathogen examination unit of any of the preceding or subsequent example combinations, wherein the application of heat occurs for less than 30 seconds, preferably less than 20 seconds, and more preferably less than 10 seconds.

EC 26. The pathogen examination unit of any of the preceding or subsequent example combinations, wherein the application of steam further comprises applying steam to the panel at a level sufficient to disinfect the panel.

EC 27. The pathogen examination unit of any of the preceding or subsequent example combinations, wherein the application of steam occurs for less than 30 seconds, preferably less than 20 seconds, and more preferably less than 10 seconds.

EC 28. The pathogen examination unit of any of the preceding or subsequent example combinations, wherein the application of chemicals further comprises applying at least one of alcohol, chlorine, glutaraldehyde, p-Chloro-o-benzylphenol, o-Phenylphenol, hydrogen peroxide, idophors, ortho-phthaladehyde or peracetic acid to the panel.

EC 29. The pathogen examination unit of any of the preceding or subsequent example combinations, wherein the application of chemicals occurs for less than 30 seconds, preferably less than 20 seconds, and more preferably less than 10 seconds.

EC 30. The pathogen examination unit of any of the preceding or subsequent example combinations, wherein the application of cold further comprises applying cryochamber disinfectant technology to the panel.

EC 31. The pathogen examination unit of any of the preceding or subsequent example combinations, wherein the application of cold occurs for less than 30 seconds, preferably less than 20 seconds, and more preferably less than 10 seconds.

EC 32. The pathogen examination unit of any of the preceding or subsequent example combinations, wherein the non-working sides may be opaque.

EC 33. The pathogen examination unit of any of the preceding or subsequent example combinations, wherein the non-working sides may extend beyond the working side to partially surround a person positioned outside the pathogen examination unit and in front of the working side.

EC 34. The pathogen examination unit of any of the preceding or subsequent example combinations, wherein the working side comprises a pair of gloves affixed and sealed to a pair of holes in the panel.

EC 35. The pathogen examination unit of any of the preceding or subsequent example combinations, wherein multiple units may be joined to each other using a common non-working side between the units.

EC 36. The pathogen examination unit of any of the preceding or subsequent example combinations, wherein common non-working side does not contain a panel.

EC 37. The pathogen examination unit of any of the preceding or subsequent example combinations, further comprising a collection unit.

EC 38. The pathogen examination unit of any of the preceding or subsequent example combinations, wherein the collection unit comprises at least one of a drawer, collection bin, or funnel.

EC 39. The pathogen examination unit of any of the preceding or subsequent example combinations, wherein the collection unit can be accessed by a patient to place a specimen or test kit inside the collection unit.

EC 40. The pathogen examination unit of any of the preceding or subsequent example combinations, wherein the collection unit can be manipulated to discharge the specimen or the test kit into a sealable container.

EC 41. The pathogen examination unit of any of the preceding or subsequent example combinations, wherein the collection unit is part of a wall of collection units.

EC 42. The pathogen examination unit of any of the preceding example combinations, wherein a cycle time of patient testing and pathogen working side cleaning is between 6.5 to 7.5 minutes.

The above-described aspects are merely possible examples of implementations, merely set forth for a clear understanding of the principles of the present disclosure. Many variations and modifications can be made to the above-described embodiment(s) without departing substantially from the spirit and principles of the present disclosure. All such modifications and variations are intended to be included herein within the scope of the present disclosure, and all possible claims to individual aspects or combinations of elements or steps are intended to be supported by the present disclosure. Moreover, although specific terms are employed herein, as well as in the claims that follow, they are used only in a generic and descriptive sense, and not for the purposes of limiting the described invention, nor the claims that follow.

That which is claimed:

1. A pathogen examination unit comprising:
a working side;
two non-working sides; and
an entrance side,
wherein the working side comprises a panel that is positioned between a healthcare worker and a patient.

2. The pathogen examination unit of claim 1, wherein the panel comprises an antimicrobial treatment.

3. The pathogen examination unit of claim 1, wherein the entrance side comprises a panel comprising a door, wherein the door forms an airtight seal through use of at least one of a magnet or a gasket.

4. The pathogen examination unit of claim 1, wherein each non-working side comprises a panel.

5. The pathogen examination unit of claim 4, further comprising an upper surface and a lower surface, wherein the working side, two non-working sides, the entrance side, the upper surface, and the lower surface are joined to each other via frame components.

6. The pathogen examination unit of claim 5, wherein each frame component comprises two channels into which an edge of two panels meeting at a perpendicular angle are inserted.

7. The pathogen examination unit of claim 5, wherein the entrance side comprises a panel comprising a door, wherein the door forms an airtight seal through use of at least one of a magnet or a gasket wherein the pathogen examination unit is sealed when the door is closed.

8. The pathogen examination unit of claim 5, further comprising a ventilation system connected to the pathogen examination unit, and wherein the ventilation system is configured to at least one of:
supply a positive air pressure inside the unit when a healthcare worker is located inside the unit; or
supply a negative air pressure inside the pathogen examination unit when a patient is located inside the unit.

9. The pathogen examination unit of claim 1, wherein the panel is cleaned by application of light, and wherein the application of light further comprises application of ultraviolet germicidal irradiation for less than 30 seconds.

10. The pathogen examination unit of claim 9, wherein a light source that provides the ultraviolet germicidal irradiation is at least one of:
positioned within the pathogen examination unit and directed through the panel; or
positioned outside the pathogen examination unit and directed onto the panel.

11. The pathogen examination unit of claim 9, wherein a light source that provides the ultraviolet germicidal irradiation provides ultraviolet light within a 200-300 nm range, and wherein the light source is at least one of ultraviolet light-emitting diodes, pulsed xenon lamps, and mercury lamps.

12. The pathogen examination unit of claim 9, wherein a light source that provides the ultraviolet germicidal irradiation is movable across a surface of the panel.

13. The pathogen examination unit of claim 1, wherein the panel is cleaned by application of light, and wherein the application of light further comprises application of lasers for less than 30 seconds, preferably less than 20 seconds, and more preferably less than 10 seconds.

14. The pathogen examination unit of claim 1, wherein the panel is cleaned by application of heat, wherein the application of heat further comprises use of a metallic grid attached to or imbedded within the panel to generate heat and raise a panel temperature to a level sufficient to disinfect the panel.

15. The pathogen examination unit of claim 1, wherein the panel is cleaned by application of steam, and wherein the application of steam further comprises applying steam to the panel at a level sufficient to disinfect the panel.

16. The pathogen examination unit of claim 1, wherein the panel is cleaned by application of chemicals, and wherein the application of chemicals further comprises applying at least one of alcohol, chlorine, glutaraldehyde, p-Chloro-o-benzylphenol, o-Phenylphenol, hydrogen peroxide, idophors, ortho-phthaladehyde or peracetic acid to the panel.

17. The pathogen examination unit of claim 1, wherein the panel is cleaned by application of cold, and wherein the application of cold further comprises applying cryochamber disinfectant technology to the panel.

18. The pathogen examination unit of claim 1, wherein the non-working sides extend beyond the working side to partially surround a person positioned outside the pathogen examination unit and in front of the working side.

19. The pathogen examination unit of claim 1, wherein the working side comprises a pair of gloves affixed and sealed to a pair of holes in the panel.

20. The pathogen examination unit of claim 1, further comprising a collection unit, wherein the collection unit is configured to be accessed by a patient to place a specimen or test kit inside the collection unit.
